(19) United States
(12) Patent Application Publication
(10) Pub. No.: US 2019/0249256 A1
(43) Pub. Date: Aug. 15, 2019

(54) METHOD FOR DETERMINING THE RISK OF DEVELOPING ARTHRITIS

(71) Applicant: Academisch Medisch Centrum, Amsterdam (NL)

(72) Inventors: Niek DE VRIES, Abcoude (NL); Paul-Peter TAK, Kortenhoef (NL); Marieke E. DOORENSPLEET, Amsterdam (NL); Paulus L. KLARENBEEK, Amsterdam (NL)

(21) Appl. No.: 16/315,122

(22) PCT Filed: Jul. 6, 2017
(86) PCT No.: PCT/EP2017/067047

§ 371 Date: Jan. 3, 2019

(30) Foreign Application Priority Data
Jul. 6, 2016 (EP) 16178281.8

(51) Int. Cl. C12Q 1/6883; C12Q 1/02

(52) U.S. Cl. CPC C12Q 1/6883; C12Q 1/025; C12Q 2600/118; C12Q 2600/166; C12Q 2600/136; C12Q 2600/112; C12Q 2600/156

(57) ABSTRACT

The invention provides a method of determining the risk of developing rheumatoid arthritis in a subject comprising the steps of determining in a biological sample from said subject the number and/or frequency of dominant BCR clones, and determining the risk of developing rheumatoid arthritis based on said number of dominant BCR clones, wherein an increase of said number of dominant BCR clones and/or a higher frequency of at least one dominant BCR clone compared to a healthy control indicates an increased risk. In a preferred embodiment, said increased risk is indicated when at least 0.5% of the total number of BCR clones is a dominant BCR clone.

### TECHNICAL FIELD OF THE INVENTION

The invention relates to methods for determining the risk of developing arthritis or for monitoring the response to a preventive treatment of arthritis in a subject. In particular it relates to methods for determining the risk of developing rheumatoid arthritis (RA).

### BACKGROUND OF THE INVENTION

Rheumatoid arthritis (RA) is a chronic autoimmune disease with unknown etiology.

Clinically manifest arthritis due to synovial inflammation is the hallmark feature of RA. However, it is not the first sign of disease, as the development of synovial inflammation may be preceded by the presence of disease-specific autoantibodies (1-3). This situation is reminiscent of that in several other immune-mediate inflammatory diseases (4-7). RA-specific autoantibodies IgM-rheumatoid factor (RF) and/or anti-citrullinated protein antibodies (ACPA) can be present up to 15 years before onset of disease (1, 8-10). Towards the onset of clinically evident arthritis the ACPA repertoire may broaden due to epitope spreading (10,11), and levels of inflammatory cytokines and chemokines may increase preceding clinically manifest arthritis (12,13). Although the presence of ACPA is highly specific for RA (14) and may precede its onset, only 28% of the autoantibody positive subjects will develop arthritis within 4 years (15). Since not all individuals that have RA-specific antibodies progress to full-blown RA, researchers have been searching for biomarkers that predict which individuals will develop RA. Several markers were informative on a population level such as a panel of autoantibodies and cytokines, as well as certain gene signatures (30-32). Recently, van der Stadt et al. (24) provided a composite model in which clinical parameters were evaluated on the level of the individual. There remains a need in the art for a biomarker that has superior predictive power compared to biomarkers evaluated so far, and/or has an increased accuracy to predict the short-term development of RA in at-risk individuals.

A biomarker which allows the identification of at risk individuals who will develop clinically evident RA in the short term, will enable development of early treatment strategies, which start treatment in a very early window of opportunity (33), possibly preventing development of arthritis.

Therefore, it is an object of the invention to provide a novel biomarker for determining the risk of developing arthritis, or for monitoring the response to a preventive treatment of arthritis in a subject.

### SUMMARY OF THE INVENTION

The invention is based on the surprising finding that an increase of dominant BCR clones in peripheral blood compared to persons not at risk was associated with arthritis development. Even when adjusted for a recently described clinical prediction rule the correlation remained intact (RR=5.0; p=0.024). When individuals progressed to arthritis or RA, dominant BCR clones disappeared from peripheral blood and appeared in synovial tissue, suggesting a direct role of these clones in disease pathogenesis.

The inventors have shown that in the earliest stages of RA, the presence of an increased number of dominant BCR clonal signatures in blood predicts onset of clinically manifest RA. This marker may be used in guiding institution of more aggressive treatment in a very early window of opportunity. Moreover, the present invention for the first time shows that during onset of the clinical manifestation of arthritis in patients, these BCR clones disappear from the blood, while they appear as dominant clones in the synovium, even in clinically non-inflamed joints. Therefore, determining the presence of BCR clones in peripheral blood or in the synovium is predictive of the development stage of clinically manifest RA.

The invention therefore provides a method for determining the risk of developing arthritis or for monitoring the response to a preventive treatment of arthritis in a subject, comprising the steps of:

(a) determining in a biological sample from said subject the number of dominant BCR clones and/or frequency of at least one dominant BCR clone(s), and

(b) determining said risk of developing arthritis or said response based on said number of dominant

BCR clones and/or frequency of said at least one dominant BCR clone(s).

Preferably, a dominant BCR clone is defined as a group of cells expressing the same BCR and wherein the amount of mRNA encoding the BCR of the cells belonging to said BCR clone constitutes at least 0.1% of the total amount of mRNA encoding a BCR in the biological sample, and wherein an increase of said number of dominant BCR clones and/or a higher frequency of at least one dominant BCR clone(s) compared to a healthy control indicates an increased risk or a poor response. In a preferred embodiment, said subject is characterized by the presence of IgM-RF and/or ACPA. Preferably, said subjects are persons “at risk”. Preferably, said level of IgM-RF is higher than 12.5 kU/L and/or said level of ACPA is higher than 25 kAU/L. In another preferred embodiment, said subject suffers from arthralgia.

In a preferred embodiment, said dominant BCR clone is defined as a group of B cells or plasma cells sharing a unique BCR signature, wherein said BCR signature of said BCR clone represents at least 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0% of the total number of unique signatures detected in the biological sample. Preferably this is determined by determining the percentage of sequences attributable to a BCR clone of the total number of BCR sequences in the biological sample.

Preferably, at least one dominant BCR clone in the biological sample is of the IgG isotype, more preferably of the IgG1 subclass.

Preferably, said biological sample is a peripheral blood sample or a synovial tissue sample. Preferably, an increased risk is indicated when at least 2, 3, 4, 5, 10 or 15 dominant clones are present in said biological sample.

In a preferred embodiment, said developing arthritis occurs within 60 months, preferably within 48 or 36, 24 or 12 months.

Preferably, said arthritis is rheumatoid arthritis (RA). Preferably, said rheumatoid arthritis is as defined according to ACR and/or EULAR criteria.

In another preferred embodiment, the method comprises the steps of:

(a) obtaining a nucleic acid from the biological sample,

(b) performing the amplification of representative sequences of the B cell receptor which enable the identification of a B cell receptor,

(c) quantifying the number and/or frequency of B cell receptor(s), and

(d) determining the number and/or frequency of dominant BCR clone(s) based on said number and/or frequency of B cell receptor(s).

Preferably, said representative sequences of the B cell receptor comprise unique VDJ and/or CDR3 sequences of the heavy chain of said B cell receptor, or VJ and/or CDR3 sequences of the light chain of the B cell receptor.

Preferably, the amplification of the nucleic acid is performed using a first primer capable of specifically hybridizing in stringent conditions with the nucleic acids selected from the group consisting of a IGHV, IGKV and IGLV, and a second primer capable of specifically hybridizing in stringent conditions with the nucleic acid selected from: (a) IGHC and IGHJ,

(b) IGKC and IGKJ, or

(c) IGLC and IGLJ.

Preferably, said method is used in combination with one or more further biomarker(s) or risk factor associated with the development of arthritis or rheumatoid arthritis, or with the response to a preventive treatment of arthritis. In a preferred embodiment, said further biomarker or risk factor is as described in Table 2 of M. H. van Beers-Tas et al., Best Practice & Research Clinical Rheumatology 29(2015)527-542.

The invention further provides a compound for use in the preventive treatment of a subject at risk of developing arthritis, wherein said risk is determined according the method to the invention. Preferably, said compound is selected from Rituximab, Etanercept, Adalimumab, Anakinra Infliximab and Abatacept. More preferred said compound targets or depletes B-cells, plasmablasts and/or plasmacells. More preferred is therapy targeting CD38, e.g. Daratumumab, Isatuximab, MOR202, Ab79, Ab19 (from Takeda), a bispecific antibody against CD3/CD38 (from Xencor), an anti-CD38 antibody-drug conjugate MT-4019 or SAR 650984, therapy inhibiting proteasome, e.g. Ninlaro, Velcade or Kyprolis, therapy inhibiting brutons kinase, e.g. Ibrutinib, acalabrutinib (ACP-196), BAY 1238097 or PRN1008, or therapy targeting CD20, e.g. Ofatumumab, Ocrelizumab or Rituximab. Most preferred is Rituximab.

### BRIEF DESCRIPTION OF THE FIGURES

FIG. 1 shows the clinical characteristics of healthy controls, at-risk individuals that didn't develop arthritis over time, and at-risk individuals that developed arthritis. At-risk individuals have elevated titers for IgM-RF (>12.5 kU/L) and/or anti-CCP (>25 kAU/L). Healthy individuals have low titers for IgM-RF (≤12.5 kU/L) and anti-CCP (≤25 kAU/L). IgM-RF=rheumatoid factor of the IgM isotype, anti-CCP=anti-cyclic citrullinated peptide antibodies, ESR=erythrocyte sedimentation rate, CRP=C-reactive protein, 68TJC=tender joint count assessed in 68 joints, 66SJC=swollen joint count assessed in 66 joints. * only in individuals who were positive, levels were categorized into high/low positive according to cut-off levels used in the 2010 ACR/EULAR criteria for RA, measured in kAU/L, § measured in mm/hr, measured in mg/L

FIG. 2(A) shows a scatterplot of the BCR repertoire in peripheral blood of 11 at-risk individuals who developed arthritis (at-risk developed arthritis), 10 at-risk individuals who didn't develop arthritis (at-risk no arthritis developed), and 10 auto-antibody negative healthy individuals. Each dot represents one clone. The size of the clones is depicted as percentage of the total BCR heavy sequences. FIG. 2(B) shows the absolute number of dominant BCR clones (clonal size ≥0.5% of the total repertoire), FIG. 2(C) shows the impact of all dominant clones combined, and FIG. 2(D) shows the size of the single most dominant clone, in at-risk individuals that developed arthritis (at-risk arthritis, n=11) versus at-risk individuals that didn't develop arthritis yet (at-risk no arthritis, n=10), and healthy individuals (healthy, n=10). Bars show mean and SD, ***p<0.0001, **p<0.001 using 1 way-ANOVA.

FIG. 3 shows the clinical characteristics of at-risk individuals that didn't develop arthritis over time, and at-risk individuals that developed arthritis. IgM-RF=rheumatoid factor of the IgM isotype, anti-CCP=anti-cyclic citrullinated peptide antibodies, ESR=erythrocyte sedimentation rate, CRP=C-reactive protein, 53TJC=tender joint count assessed in 53 joints. * only in individuals who were positive, † levels were categorized into high/low positive according to cut-off levels used in the 2010 ACR/EULAR criteria for RA because of changed reference values over time, ¶ measured in kAU/L, § measured in mm/hr, ‡ measured in mg/L, º score based on the risk rule (24) scaled 0 to 13 points.

FIG. 4(A-F) shows the Receiver Operating Characteristic (ROC) curves for (A) the number of dominant clones, (B) the impact of all dominant clones combined, and (C) the impact of the most dominant clone, in at-risk individuals (n=21). The development of arthritis was analyzed after 36 months of follow-up. The arrow points to the cut-off value chosen, and the corresponding value is shown. AUC=area under the curve. (D) Kaplan-Meier curve for BCR-clone positive and BCR-clone negative individuals in the initial cohort, assuming the at-risk individuals analyzed represent a random selection of the total at-risk individuals (n=65). (E) Kaplan-Meier curve for BCR-clone positive and BCR-clone negative individuals in the replication cohort. (F) Table describing sensitivity, specificity, positive predicting value (PPV) and negative predicting value (NPV) including 95% confidence intervals for the BCR-clone model, in the initial cohort, the replication cohort, and these 2 cohorts combined.

FIG. 5(A/B) shows a scatter plot of the BCR repertoire in synovial tissue (A) and peripheral blood (B) after arthritis development (n=8). Each dot represents one clone. Large dots represent clones that were dominantly present in in peripheral blood during the pre-clinical phase. The size of the clones is depicted as percentage of the total BCR_{heavy} sequences. FIG. 5(C) shows a dot plot showing the overlap between dominant BCR clones in the preclinical phase and after arthritis development (n=8). The y-axis depicts the rank of the clones found in blood during the pre-clinical phase (all 8 patients pooled). On the left x-axis the overlap with dominant clones in peripheral blood after arthritis development, on the right x-axis the overlap with dominant clones in synovial tissue after arthritis development. In case no overlap was found, the dots were marked ‘no overlap’.

FIG. 6 shows the clinical characteristics of individuals that were and individuals that were not included in example I

FIG. 7 shows ROC curves of the number of dominant clones. Follow-up was divided in 5 different groups, and depicted separately. The black dots show the sensitivity and specificity for the presence of 5 dominant clones.

FIG. 8 (A) shows the percentage of the 25 most dominant clones occupied by the different immunoglobulin isotypes in peripheral blood during the preclinical phase, in 21 at-risk individuals, of which 11 developed arthritis (at-risk arthritis developed), and 10 didn't developed arthritis yet (at-risk no arthritis), and 10 healthy individuals as controls (healthy individuals). Bars show mean and SD, ***p<0.0001 using two-way ANOVA. FIG. 8 (B) shows the percentage of the IgG+ clones occupied by the 4 subtypes in peripheral blood during the preclinical phase. Bars show mean and SD, ***p<0.0001 using two-way ANOVA. FIG. 8 (C) shows the percentage of total clones that are IGHV4-34 bearing in the 25 most dominant clones in peripheral blood during the preclinical phase. Mean and IQR are depicted, *p<0.05 using Tway-ANOVA). (D) CDR3 length of dominant clones in peripheral blood. All bars show mean and SD (*p=0.05, **p<0.01, ***p<0.001 using 1 way-ANOVA).

FIG. 9 shows that in the individuals that developed arthritis, the time to arthritis is correlated with the number of expanded BCR clones (HECs) present in the biological sample.

FIG. 10 shows the correlation between the total impact of dominant BCR clones (% of the total BCR repertoire) and the time to develop arthritis.

FIG. 11 shows the correlation between the impact of the most dominant BCR clone and the time to develop arthritis.

FIG. 12 shows for individuals having a given number of hecs or more respectively the number of individuals having arthritis at three years, the total number of individuals, the number of individuals that did not develop arthritis, the consequent risk for arthritis at three years given the number of hecs, the number of patients developing arthritis during follow-up and the average number of months until arthritis.

### DETAILED DESCRIPTION OF THE INVENTION

Definitions

The term “arthritis” refers to a disease characterized by the inflammation of the membrane lining the joint, which causes pain, stiffness, warmth, redness or swelling.

As used herein, “ rheumatoid arthritis” or “RA” refers to an autoimmune disease that causes chronic inflammation of the joints, the tissue around the joints, as well as other organs in the body. Preferably, a diagnosis is established by a physician. Preferably, rheumatoid arthritis is diagnosed using the ACR and/or EULAR criteria (Aletaha D et al., Arthritis Rheum. 2010 Sep;62(9):2569-81).

The term “pre-clinical RA” as used herein refers to a phase preceding the onset of RA, characterized by the presence of specific autoantibodies, in the absence of clinically evident synovial inflammation. Pre-clinical RA can usually be determined with certainty after a definitive diagnosis of RA has been established. In that context pre-clinical RA represents the phase preceding the onset of arthritis.

The term “at risk of RA” or “at risk of developing RA” as used herein refers to subjects suffering from arthralgia and have detectable levels of autoantibodies in their blood, in particular IgM-RF and/or ACPA antibodies. As used herein, “preventive treatment” of a disease-state in a subject, and include: (a) preventing the disease-state from occurring in a subject, in particular, when said subject is predisposed to the disease-state but has not yet been diagnosed as having it; (b) inhibiting the disease-state, i.e., arresting it development; and/or (c) relieving the disease-state, i.e., causing regression of the disease state.

The term “B cell receptor” or “BCR” refers to a specialized transmembrane receptor protein located on the outer surface of B cells. The receptor's binding moiety is composed of a membrane-bound antibody that, like all antibodies, has a unique and randomly determined antigen-binding site.

The term “B cell clone” or “BCR clone” as used herein refers to a group of B cells or plasma cells sharing the same unique BCR signature or expressing the same B cell receptor. Preferably, B cells, plasmablasts or plasma cells belonging to a certain BCR clone express the same unique VDJ and/or CDR3 rearrangement of the heavy chain encoding the heavy chain of a B cell receptor. In another preferred embodiment, the cells belonging to a certain BCR clone express the same VJ and/or CDR3 rearrangement of the light chain. In an embodiment, the cells belonging to a certain BCR clone expressing the same heavy and light chain have the same antigen specificity.

The term “frequency of a BCR clone” as used herein refers to the relative number, including a percentage or a fraction of a larger population of cells belonging to a certain BCR clone. A BCR clone is considered more abundant compared to another B cell clone in case said BCR clone has a higher frequency when compared to said other BCR clone isolated from the same or a comparable biological sample. For the sake of completeness, the frequency of a BCR clone can be expressed as a percentage of the total BCR repertoire, by dividing the number of times that this clone's unique BCR signature is detected over the total number of individual signatures detected in the biological sample and then multiplying by 100. E.g. if an unique BCR signature is detected 100 times in a sample, and a total of 10.000 individual BCR sequences are detected, the frequency as a percentage is 100/10000×100=1% in the particular sample.

The term “BCR signature” or “signature” as used herein refers to the molecular identifier identifying an individual cell expressing a BCR. Each cell belonging to a BCR clone typically has one BCR signature. A BCR signature may be determined using a test which identifies characteristics of a B-cell receptor, e.g. the CDR3 sequence and/or somatic hypermutations in the V-regions, that identify a clone of plasma cells, plasmablasts and/or B cells. Based on such a test, said number of cells belonging to a BCR clone may be determined by counting signals representative of the BCR signals. Therefore, in a preferred embodiment, said dominant BCR clone is a B cell clone wherein the number of specific signatures from this clone constitutes at least 0.1, 0.2, 0.3, 0.4, 0.5, 0.6, 0.7, 0.8, 0.9, 1.0, 1.1, 1.1, 1.2, 1.3, 1.4, 1.5, 1.6, 1.7, 1.8, 1.9 or 2.0% of the total number of signatures from all BCR clones in the biological sample.

The term “BCR repertoire” of a certain biological sample as used herein refers to the ensemble of different BCR signatures detected in the biological sample.

The term “BCR sequence” as used herein refers to a nucleic acid encoding the BCR. The term “Next-generation sequencing” (NGS) as used herein refers to sequencing technologies that have the capacity to sequence polynucleotides at speeds that were unprecedented using conventional sequencing methods (e.g., standard Sanger or Maxam-Gilbert sequencing methods). These unprecedented speeds are achieved by performing and reading out millions of sequencing reactions in parallel. NGS platforms include, but are not limited to, the following: Massively Parallel Signature Sequencing (Lynx Therapeutics); 454 pyro-sequencing (454 Life Sciences/Roche Diagnostics); solid-phase, reversible dye-terminator sequencing (Solexa/Illumina); SOLiD technology (Applied Biosystems); Ion semiconductor sequencing (Ion Torrent); and DNA nanoball sequencing (Complete Genomics). Descriptions of certain NGS platforms can be found in the following: Shendure, et al., “Next-generation DNA sequencing,” Nature, 2008, vol. 26, No. 10, 1135-1145; Mardis, “The impact of next-generation sequencing technology on genetics,” Trends in Genetics, 2007, vol. 24, No. 3, pp. 133-141; Su, et al., “Next-generation sequencing and its applications in molecular diagnostics” Expert Rev Mol Diagn, 2011, 11(3):333-43; “Sequencing technologies the next generation” by Michael Metzker, Nat Rev Genetics January 2010; and Zhang et al., “The impact of next-generation sequencing on genomics”, J Genet Genomics, 2011, 38(3):95-109.

The term “IGHV” refers to the ImmunoGlobulin Heavy chain Variable cluster (IGHV @, encoded on chromosome 14q32.33, MIM:147070 accessible via http://omim org/entry/147070) encoding the V-genes of the heavy chain.

The term “IGHJ” refers to refers to the ImmunoGlobulin Heavy chain Joining gene cluster (IGHJ@, encoded on chromosome 14q32.33, MIM:147010 accessible via http://omim org/entry/147010) encoding the J-genes of the heavy chain.

The term “IGHC” refers to the Immunoglobulin Heavy chain Constant region, being the loci encoding the alpha (IGHA), delta (IGHD), epsilon (IGHE), gamma (IGHG) and mu (IGHM) globulins, and their individual subtypes (all encoded on chromosome 14q32.33, MIMs:146900, 147000, 146910, 147170, 147180, 147100, 147110, 147120, 147130, 147020, accessible via http://omim.org).

The term IGKC refers to the Immunoglobulin Kappa Light chain Constant region, encoded on chromosome 2p11.2, MIM:147200 accessible via http://omim org/entry/147200) encoding the C-genes of the kappa light chain.

The term IGKJ refers to the Immunoglobulin Kappa Light chain Joining gene cluster IGKJ@, encoded on chromosome 2p12, MIM:146970 accessible via http://omim.org/entry/146970) encoding the J-genes of the kappa light chain.

The term IGKV refers to the Immunoglobulin Kappa Light chain Variable gene cluster IGKV @, encoded on chromosome 2p12, MIM:146980 accessible via http://omim.org/entry/146980) encoding the V-genes of the kappa light chain.

The term IGLC1 refers to the Immunoglobulin Lambda Light chain Constant region, encoded on chromosome 22q11.22, MIM:147220 accessible via http://omim.org/entry/147220) encoding the C-genes of the lambda light chain.

The term IGLJ refers to the Immunoglobulin Lambda Light chain Joining gene cluster IGLJ@, encoded on chromosome 22q11.2, MIM:147230 accessible via http://omim.org/entry/147230) encoding the J-genes of the lambda light chain.

The term IGLV refers to the Immunoglobulin Lambda Light chain Variable gene cluster IGLV @, encoded on chromosome 22q11.2, MIM:147240 accessible via http://omim.org/entry/147240) encoding the V-genes of the lambda light chain.

The term “biomarker associated with the development of arthritis or with the response to a preventive treatment of arthritis” refers to any known biomarker of risk factor, including but not limited to weight, smoking, HLA-status, serological status, cytokine profiles, CRP etc.

Dominant Peripheral Blood BCR Clones and Onset of Arthritis

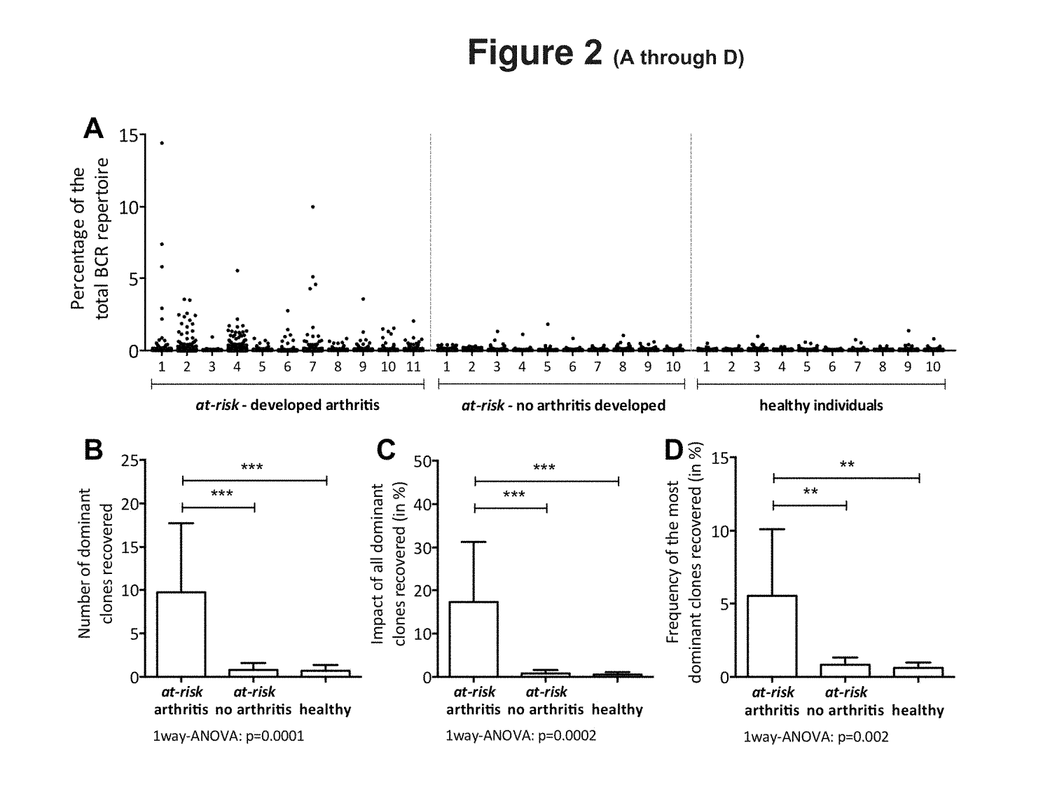

The invention is based on the finding that multiple dominant BCR clones were detected in peripheral blood of 11 prospectively followed at-risk individuals that develop arthritis, as long as 66 months before the clinical onset of arthritis. In contrast, dominant BCR clones were almost absent both in at-risk individuals that did not develop arthritis and in healthy individuals (FIG. 2A). The inventors observed that the number of dominant BCR clones, the impact of all dominant BCR clones combined, and the impact of the most dominant BCR clone was increased in at-risk individuals that developed arthritis, compared to at-risk individuals that did not develop arthritis and healthy individuals (number of dominant clones mean 9.7±8.0 vs. 0.8±0.8 vs. 0.7±0.7 respectively, p=0.001 (FIG. 2B), impact of the dominant clones on the repertoire median 16.4%, IQR 3.7-33.7% vs. 0.7% IQR 0-1.7% vs. 0.5% IQR 0-1.1% respectively, p<0.0001 (FIG. 2C) and impact of the single most dominant clone mean 5.5%±4.6% vs. 0.7%±0.7% vs. 0.6%±0.4% respectively, p<0.0003 (FIG. 2D)). Subsequently, the inventors analyzed synovial tissue biopsies in at-risk individuals in the pre-clinical phase, but these samples contained too little BCR mRNA for NGS, which is in line with the absence of B-cells that was observed with IHC (3).

Collectively, these observations demonstrate that expanded BCR clones are readily detectable in peripheral blood during the pre-clinical phase in at-risk individuals that will develop RA, but not in those that will not.

The Presence of Dominant BCR Clones Predicts Arthritis Development

In the initial cohort the presence of dominant clones was associated with onset of arthritis in at-risk individuals. Based on these data the inventors aimed to develop a clinical marker that can be used to identify individuals that have a high risk of developing arthritis in the short term. Such patients might be treated in the at-risk phase to prevent onset of arthritis. A clinically relevant follow-up period of thirty-six months was chosen to evaluate arthritis development. In their view this time period carries high enough risk to justify intervention, while being short enough to infer urgency for treatment.

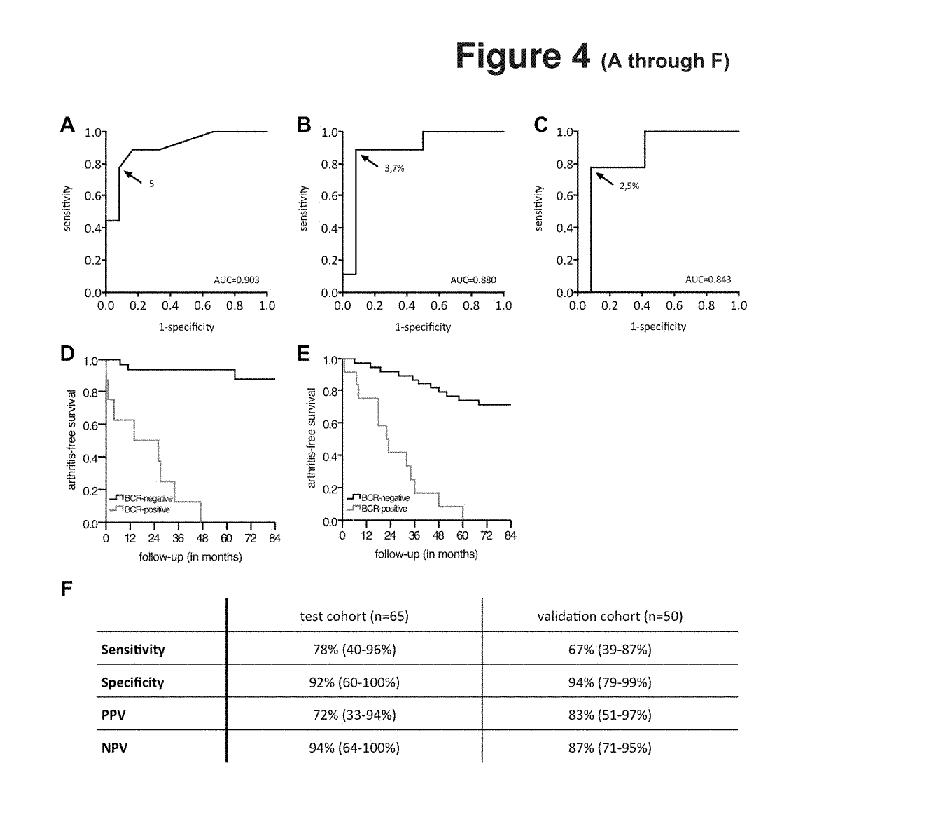

The inventors designed three tests based on the number of dominant BCR clones present, the impact of all dominant clones combined on the BCR repertoire, and the impact of the single most dominant BCR clone (FIG. 2B-D); Receiver Operating Characteristic (ROC) curves are depicted in FIG. 4A-C. Based on these ROC curves and most importantly, the positive predictive value, optimal cut-offs were determined at dominant BCR clones in peripheral blood, a combined impact 3.7%, and an impact of the most dominant clone 2.5% respectively. the inventors decided to use the presence of dominant BCR clones as biologically most relevant for further studies (further denoted as ‘BCR-positive’ , and <5 dominant BCR clones as ‘BCR-negative’, and collectively as the BCR-clone model).

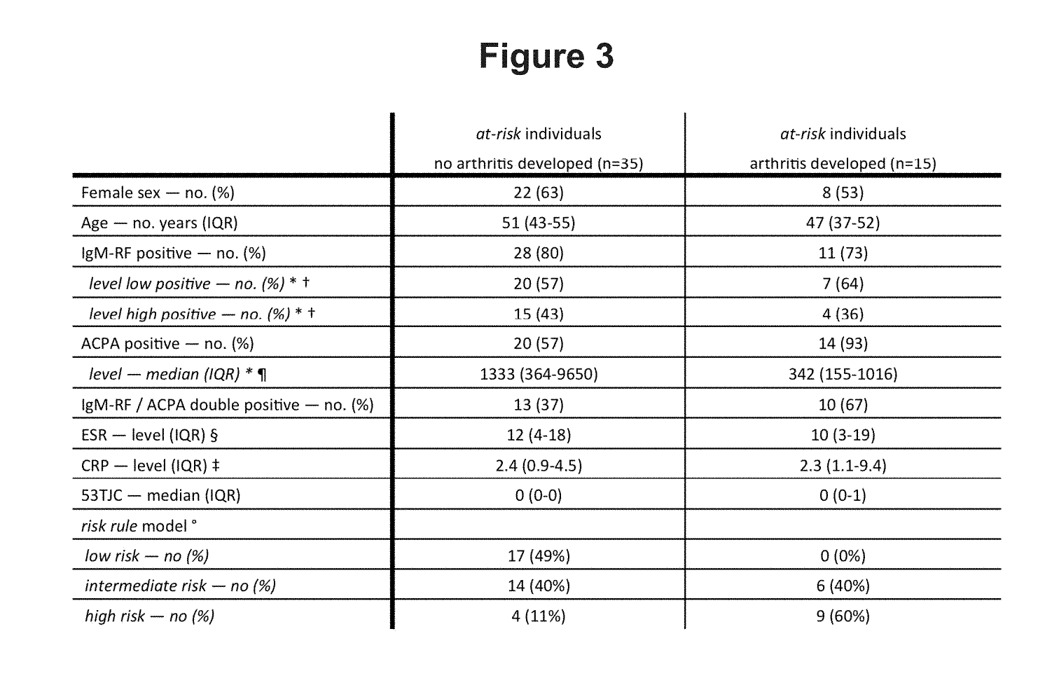

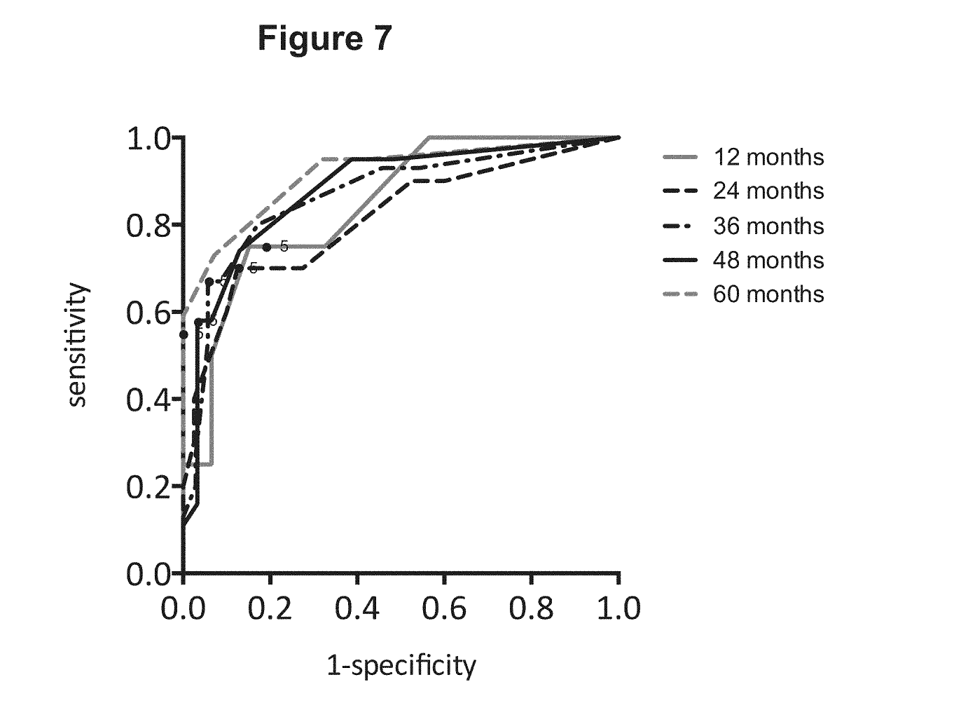

The cut-off of dominant clones in peripheral blood resulted in two clearly distinguishable groups, and corresponding sensitivity of 78% and specificity of 92% (FIG. 4F, and FIG. 4D for the Kaplan-Meier curve). The inventors replicated this analysis in a second independent prospective cohort of 50 patients, using this same cut-off (15 at-risk individuals developed arthritis within 36 months, characteristics are described in FIG. 3). In this cohort, at risk individuals who developed arthritis again showed a clear increase in the number of dominant clones compared to at-risk individuals that did not develop arthritis (mean 5.5±3.4 vs. 1.4±1.8 respectively, p<0.0001, not depicted; Kaplan-Meier curve FIG. 4E). At-risk BCR-clone positive individuals had a 83% risk of developing RA within 36 months, while this risk was 13% in at-risk BCR-clone negative individuals, implicating a relative risk of 6.3 (95%-CI 2.7-15, p<0.0001). Post-hoc analysis on the follow-up time revealed that extending the follow-up time to 60 months resulted in a specificity of 100% (in total, 3 at-risk individuals with ≥5 clones developed arthritis after 47, 48 and 60 months, FIG. 7).

The 50 at-risk individuals in the replication cohort were recently used in a prediction model for the development of RA (24), the risk rule model. This describes a composite score of multiple clinical parameters dividing at-risk individuals into low, intermediate and high risk individuals (respectively 17, 20 and 13 individuals). A logistic regression analysis on these 50 patients for both models combined showed that the BCR-clone model had added value compared to the risk rule (p=0.009) with an overall relative risk of 5.0 (95% CI 1.2-20). In the low, intermediate and high risk groups the relative risk contributed by BCR clone positivity was estimated at 18 (0.6-520), 6.1 (1.9-20) and 1.2 (0.6-2.7) respectively.

In conclusion, the inventors have shown that at-risk individuals with 5 or more dominant BCR clones in peripheral blood have a 77% risk of developing arthritis within 36 months, compared to a risk of 9% in individuals with 4 or less dominant BCR clones.

Dominant BCR Clones In Peripheral Blood In the Preclinical Phase Have Migrated To Synovial Tissue After Development of Arthritis

The inventors hypothesized that if dominant BCR clones are involved in synovial inflammation, that these clones should also be detectable in synovial tissue after onset of RA. To this end the inventors analyzed peripheral blood samples at the preclinical stage, and paired blood and synovial tissue after onset of arthritis in 8 individuals that developed arthritis.

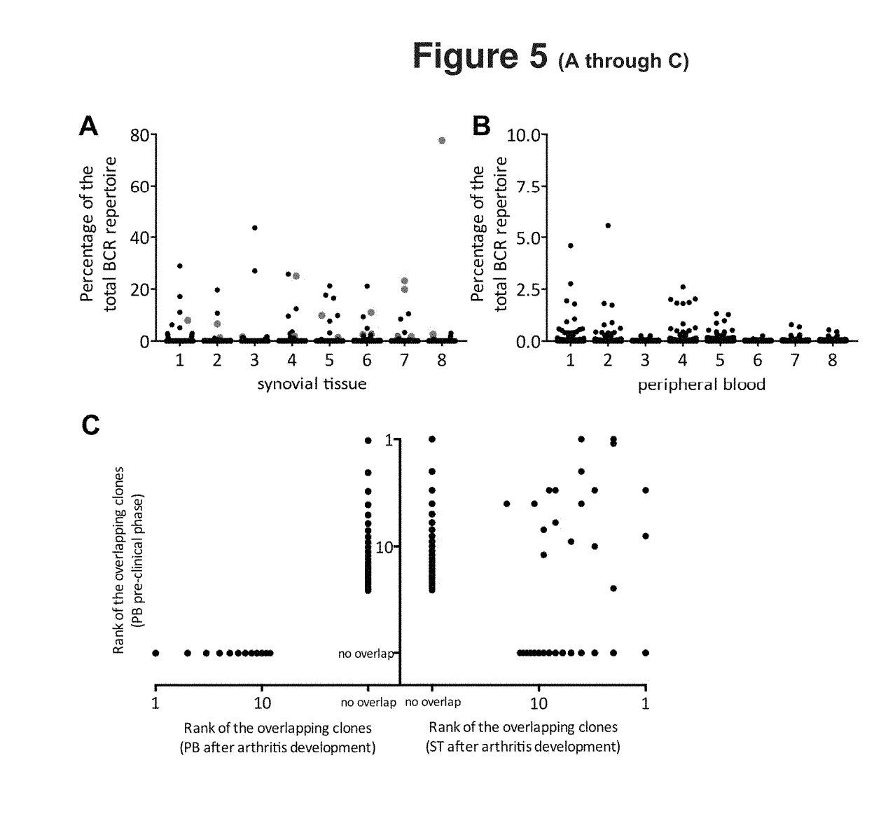

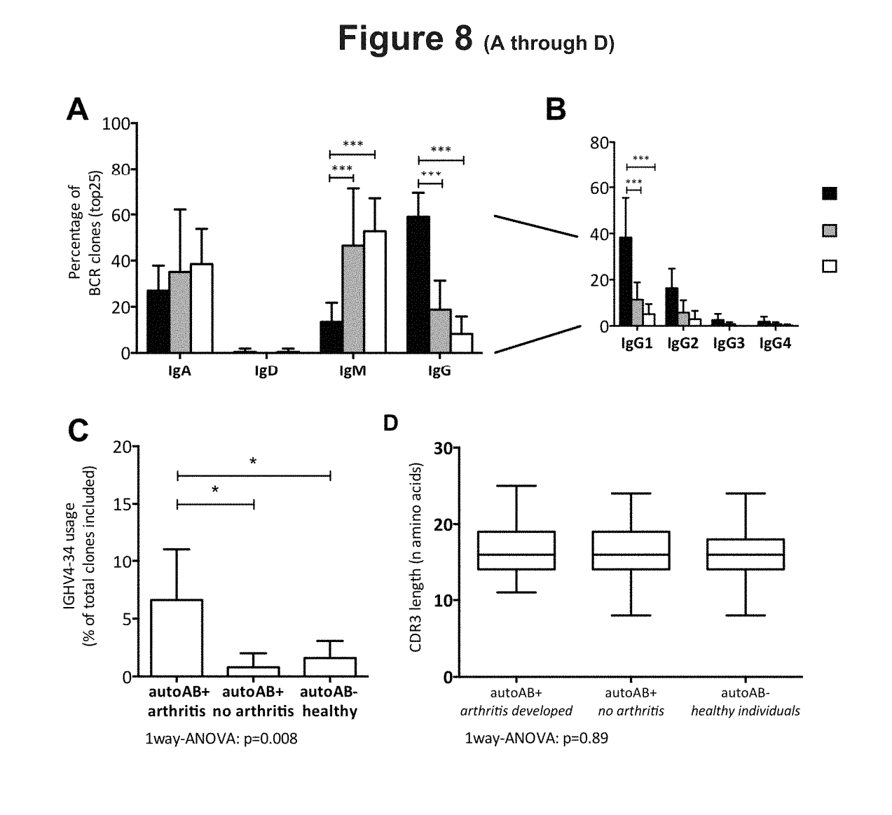

On average, 29% of all pre-clinically dominant peripheral blood BCR clones were detectable in synovial tissue after development of arthritis. All ranked within the top-25 most dominant clones in synovial tissue (FIG. 5A/C). Most strikingly, none of the pre-clinically dominant peripheral blood BCR clones could be recovered from peripheral blood after arthritis developed (FIG. 5B/C). Additional analyses of the dominant clones found in both peripheral blood and synovial tissue showed that these clones are class-switched to the IgG1 isotype and enriched for IGHV4v-34, while CDR3 lengths are comparable between the 3 groups (extensively described in supplementary results and FIG. 8).

Together, these analyses show that dominant BCR clones in peripheral blood during the preclinical phase are partially retrievable as dominant clones in synovial tissue once arthritis becomes apparent. At this time point the clones are no longer found in peripheral blood anymore. These migratory clones have features that have been associated with autoreactivity.

### Embodiments

Method For Prognosticating the Risk of Developing Arthritis or Monitoring the Response To A Treatment of Arthritis

The invention provides a method for determining the risk of developing arthritis or for monitoring the response to a preventive treatment of arthritis in a subject, comprising the steps of:

(a) determining in a biological sample from said subject the number of dominant BCR clones and/or frequency of at least one dominant BCR clone(s), and

(b) determining said risk of developing arthritis or said response based on said number of dominant BCR clones and/or frequency of said at least one dominant BCR clone(s).

Said biological sample may be any biological sample containing cells having a BCR, including B cells plasma cells and/or plasmablasts. Said biological sample includes, but is not limited to blood, synovial fluid, lymphoid tissue, bone marrow, cerebrospinal fluid, bronchoalveolar lavage fluid, saliva and peritoneal cavity fluid. Preferably, said biological sample is a peripheral blood sample. It is important that said biological sample contains a sufficient number of cells having a BCR in order to achieve statistically reliable results. Therefore, it is preferred that said biological sample contains at least 1000 cells having a BCR. Even more preferably at least 2000, 3000, 4000, 5000, 10.000, 25,000, 50,000, 100,000, 250,000, 500,000, 1,000,000 or 5,000,000 cells having a BCR. To reliably estimate the number of dominant BCR clones in a sample a minimal sample size is required.

The inventors have found that the number and/or frequency of the most expanded BCR clones are most indicative of an increased risk of developing arthritis or poor response to a preventive RA treatment. The inventors have determined that BCR clones which are expanded to a number of cells which has a total amount of mRNA encoding the BCR which constitutes at least 0.1% of the total amount of mRNA encoding a BCR in the biological sample, are BCR clones which are particularly predictive of an increased risk or poor response. Such expanded BCR clone is defined as a “dominant BCR clone” herein. The number which such dominant BCT clone comprises may depend on the relative amounts of plasma cells, plasmablasts and b cells which belong to said BCR clone, as the amount of mRNA may differ between these cell types. It is therefore preferable to determine the relative amount of mRNA encoding the BCR of said dominant BCR clone compared to the total amount of mRNA encoding a BCR of all cells having a BCR in the biological sample.

It is preferred to determine the risk or response to the preventive treatment on the number and/or frequency of more expanded BCR clones, of which the combined mRNA encoding the BCR of said clone is at least 0.2, 0.3, 0.4, 0.5, 0.6, 0.7, 0.8, 0.9, 1.0, 1.1, 1.2, 1.3, 1.4, 1.5, 1.6, 1.7, 1.8, 1.9 or 2.0% of the total amount of mRNA encoding a BCR in the biological sample.

It is not required that the amount of mRNA encoding a BCR is determined directly. In principle, any method for determining the number of BCR clones in a biological sample may be used.

The method of the invention encompasses any method wherein the relative size of a BCR clone is determined. For instance, the number of cells belonging to a certain BCR may be determined and compared to the number of cells belonging to other BCR clones within the biological sample or compared with the total number of cells having a BCR within the biological sample. In such case, the cut-off value for establishing whether a certain BCR clone is a dominant BCR clone may be suitably be determined by establishing how many cells contribute to a certain amount of mRNA which encodes a BCR. For example, in a reference sample with a known amount of mRNA encoding the BCR of a certain BCR clone, the number of cells belonging to said BCR clone may be determined by cell counting and the number of cells of said clone may be compared to the overall number of cells having a BCR within the sample.

A skilled person may use other parameters to determine the relative size or frequency of a BCR clone to determine whether it is a dominant BCR clone, based on the information provided herein. For example, a skilled person may select a BCR clone having an amount of BCR mRNA of 0.1% the total amount of BCR mRNA in a biological sample. The skilled person can determine the relative number of cells of this particular BCR clone or the relative amount of DNA encoding the BCR of this clone. It is therefore possible to determine whether a particular BCR clone is a dominant BCR clone as defined herein, based on relative cell number, the relative amount of DNA encoding the BCR compared to the total amount of DNA encoding a BCR in the biological sample and so on.

An increase in the number of said dominant BCR clones in comparison to the number in a healthy control is indicative of an increased risk. To achieve a good sensitivity or specificity of the method, it is preferred that at least 2, 3, 4, 5, 6, 7, 8, 9, 10, 11, 12, 13, 14, 15, 16, 17, 18, 19, or 20 dominant BCR clones are present in the biological sample.

Preferably, an increase in the frequency of a dominant BCR clone in comparison to the frequency of a dominant BCR clone in a healthy control is also indicative of an increased risk or poor response to a preventive treatment of arthritis.

In another preferred embodiment, an increased risk or poor response is indicated if the combined unique BCR signatures of all BCR clones present in said biological sample represents at least 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 3,7%, or 5.0% of the total number of individual BCR signatures detected in the biological sample.

In another preferred embodiment, an increased risk or poor response is indicated if the amount of BCR signatures of the most numerous BCR clone present in said biological sample represents at least 1% , 2.5%, or 5% of the total number of BCR signatures detected in the biological sample.

In another preferred embodiment, an increased risk or poor response is indicated if the percentage of combined cells belonging to the all dominant BCR clones in said biological sample represent at least 1% , 5% 10%, or 20%, of the total number cells expressing a BCR in the biological sample.

In another preferred embodiment, the frequency of a dominant BCR clone is compared to the frequency of the total number of BCR clones of the IgG type or to the total number of BCR clones in general. It is preferred that in order to achieve a good sensitivity and specificity of the method that at least 1, 2, 3, 4, 5, 6, 7, 8, 9, 10% of the total number of IgG BCR clones is a dominant BCR clone.

In another preferred embodiment, the frequency of a dominant BCR clone is compared to the frequency of the total number of BCR clones of the IgA type or to the total number of BCR clones in general. It is preferred that in order to achieve a good sensitivity and specificity of the method that at least 1, 2, 3, 4, 5, 6, 7, 8, 9, 10% of the total number of IgA BCR clones is a dominant BCR clone.

In another preferred embodiment, the frequency of a dominant BCR clone is compared to the frequency of the total number of BCR clones of the IgM type or to the total number of BCR clones in general. It is preferred that in order to achieve a good sensitivity and specificity of the method that at least 1, 2, 3, 4, 5, 6, 7, 8, 9, 10% of the total number of IgM BCR clones is a dominant BCR clone.

In a preferred embodiment, at least one, preferably 2, 3, 4 or 5 of the 25 most abundant BCR clones is a dominant BCR clone. Even more preferred is a method according to the invention, wherein an increased risk is indicated when at least one, preferably 2, 3, 4 or 5 of the 10 most abundant BCR clones is a dominant BCR clone. In a preferred embodiment, it is preferred that at least 500 different BCR clones are analyzed, as this results in improved sensitivity and/or specificity.

In a preferred embodiment, an increased risk or poor response is indicated when at least 2, 3, 4 or 5 dominant BCR clones are present in the sample.

In a preferred embodiment, said at least one dominant BCR clone comprises at least 3, 4 or 5, 6, 7, 8, 9, 10, 11, 12, 13, 14, 15, 16, 17, 18, 19 or 20 dominant clones. This identifies more patients prone to develop arthritis.

Using a higher number of dominant BCR clones as a marker for determining the risk or poor response decreases the risk of false positive predictions and reduces the time to onset of arthritis. In a preferred embodiment, the presence of at least 5 dominant BCR clones is used to create an optimal balance between the fraction of not identified at-risk individuals that developed arthritis, and thus not treated, versus the additional burden of unnecessary treatment in individuals who do not develop arthritis in the immediate future. The inventors have shown that subjects with 5 or more dominant BCR clones in peripheral blood have a 83% risk of developing arthritis within 36 months.

In a preferred embodiment, said developing RA occurs within 100, more preferably within 60 months, within 48 or 36 months. It is of interest that all three patients who were BCR-positive and did not develop arthritis at 36 months developed arthritis at 47, 48 and 60 months of follow-up, indicating that treatment may be necessary for all BCR-positive subjects.

Determining The Number and Frequency of A BCR Clone

Any method to determine the number or frequency of BCR clones may be used. Such methods are well known in the art and described for instance by Gur Yaari and Steven H. Kleinstein, Genome Med. 2015; 7:121. The number of different B cell receptors in a biological sample indicates the number of different BCR clones in the sample. The number of different BCR clones can therefore be established by any method which identifies the B cell receptor of a certain clone. This will often include the rearranged nucleotide sequences comprising the CDR3 region, but may also include other parts of the heavy-chain or light-chain encoding said B cell receptor. It may preferably include a part thereof which enables the identification of the BCR clones of the IgG subclass and even more preferably the IgG1 subclass. In a preferred embodiment, the nucleic acid sequence of at least the CDR3 region is determined.

In an alternative embodiment, the number of BCR clones is determined by performing a cell-sort of IgG, IgA or IgM positive cells using anti-human IgG, IgA or IgM specific antibodies (such as described in Shen PUF, Fuller S G, Rezuke W N, Sherburne B J, DiGiuseppe J A. Laboratory, morphologic, and immunophenotypic correlates of surface immunoglobulin heavy chain isotype expression in B-cell chronic lymphocytic leukemia. Am J Clin Pathol 2001; 116:905-12), preferably by FACS, and preferably establish the nucleic acid sequence of at least the CDR3 region of those cells. The number of different CDR3 nucleic acid sequences identified indicates the number of BCR clones within the IgG, IgA or IgM population, respectively. Suitable PCR methods are described for example in US2002/0110807 A1, examples 1-3.

In an another embodiment, B-cells and plasma cells of a biological sample are sorted (preferably by FACS) as described above, followed by mRNA isolation of these B-cells and plasma cells and transcriptome sequencing, preferably using a next-generation sequencing platform. The transcriptome sequences that include at least the CDR3 region, or a part thereof which enables the identification of the BCR clones, are then collected, thus providing a list of the individual BCR sequences detected in the biological sample. This list of identified BCR sequences forms the BCR repertoire of the biological sample. By assessing the frequency of individual unique BCR sequences in the BCR repertoire, the frequency of unique BCR clones in said biological sample is determined.

Any method to establish whether a BCR clone is of the IgG1 subclass may be used. A B-cell receptor of the IgG1 subclass can suitably be detected based on the nucleic acid sequence of the CH1 domain of the C-gene segment of the heavy chain (as published online at e.g. http://www.imgt.org/IMGT_GENE-DB/GENElect?livret=0).

In a preferred embodiment of the invention, the method comprises the steps of obtaining either the cDNA from the mRNA expressed from the biological sample or the genomic DNA extract of the biological sample. Subsequently, the obtained cDNA or the genomic DNA extract is subjected to amplification using a set of IGHV, IGKV or IGLV forward primers capable of specifically hybridizing in stringent conditions with the nucleic acids encoding the variable gene segments (VH) of immunoglobulin heavy chains and a IGHC, IGKC, IGLC, IGHJ, IGKJ or IGLJ reverse primer capable of specifically hybridizing in stringent conditions with the nucleic acid encoding the respective constant or joining segment. Preferably, a primer set hybridizing to the JH or CH gene segments is used to amplify all possible immunoglobulin isotypes. An advantage thereof is that sequence information of all BCR clones is gathered, which can be used to determine the frequency of a specific B cell clone within the total number of BCR clones. For genomic DNA a JH primer is preferred over a CH primer.

Amplification of the BCR heavy or light chain can be performed by any method known in the art.

In another preferred embodiment, amplification of the nucleic acid is performed using a first primer capable of specifically hybridizing in stringent conditions with the nucleic acids selected from the group consisting of a (a) IGHC and IGHJ, (b) IGKC and IGKJ, or (c) IGLC and IGLJ, and a second primer capable of specifically hybridizing in stringent conditions with the nucleic acid selected from IGHV, IGKV and IGLV.

In another preferred embodiment, the amplification of the nucleic acid is performed using a RACE protocol starting from one or more a primer(s) capable of specifically hybridizing in stringent conditions with the nucleic acids selected from the group consisting of a (a) IGHC and IGHJ, (b) IGKC and IGKJ, or (c) IGLC and IGLJ. Preferably said RACE protocol is performed as described in Schaefer B C. Anal Biochem. 1995 May 20; 227(2):255-73. PubMed PMID: 7573945) In another preferred embodiment, RNA or DNA sequences might be directly sequenced without prior amplification.

Preferably, said amplification step is followed by Next-Generation Sequencing of the amplified nucleotides comprising at least the CDR3 region or a part thereof which enables the identification of the BCR clones. Based on the number of unique CDR3 sequences, the number of unique BCR clones can be established. Based on the number of unique BCR clones and the total number of unique BCR clones, the frequency of said BCR clones can be determined. An increase in the frequency of BCR clones in the total number of BCR clones in a biological sample of a subject compared to the frequency of BCR clones in a healthy control indicates a higher risk. It is preferred that also the frequency of the unique BCR clones is determined. The inventors have found that the presence of several highly abundant BCR clones is indicative of a higher risk of developing RA. The frequency of a unique BCR clone is preferably determined by determining the amount of amplified sequences of a specific BCR clone and compare said number with the total number of amplified sequences, preferably of all BCR clones.

In a preferred embodiment, said method of the invention is performed comprising the following steps. The obtained cDNA or the genomic DNA is subjected to linear amplification of the complete immunoglobulin heavy- or light-chain repertoire using a primer set covering all functional IGHV, IGKV or IGLV genes of the B-cell receptor. Optionally, amplified products are purified, preferably using AMPure XP SPRI-beads (#A63881, Agencourt-Bioscience, Beverly, Mass., USA), preferably in a template:bead ratio of around 1:1. Sequencing is preferably performed on a next-generation sequencing platform (for example on a Roche Genome Sequencer FLX using the Titanium platform). For each sample preferably at least 40,000 (bead-bound) BCR sequences are analyzed. If amplification is performed, a lower number of BCR sequences may be used, for instance 500 or more.

The above disclosure generally describes the present invention. Unless defined otherwise, all technical and scientific terms used herein have the same meaning as those commonly understood by one of ordinary skill in the art to which this invention belongs. A more complete understanding can be obtained by reference to the following specific examples which are provided herein for purposes of illustration only, and are not intended to limit the scope of the invention.

### EXAMPLE SECTION

### Methods

Study Subjects

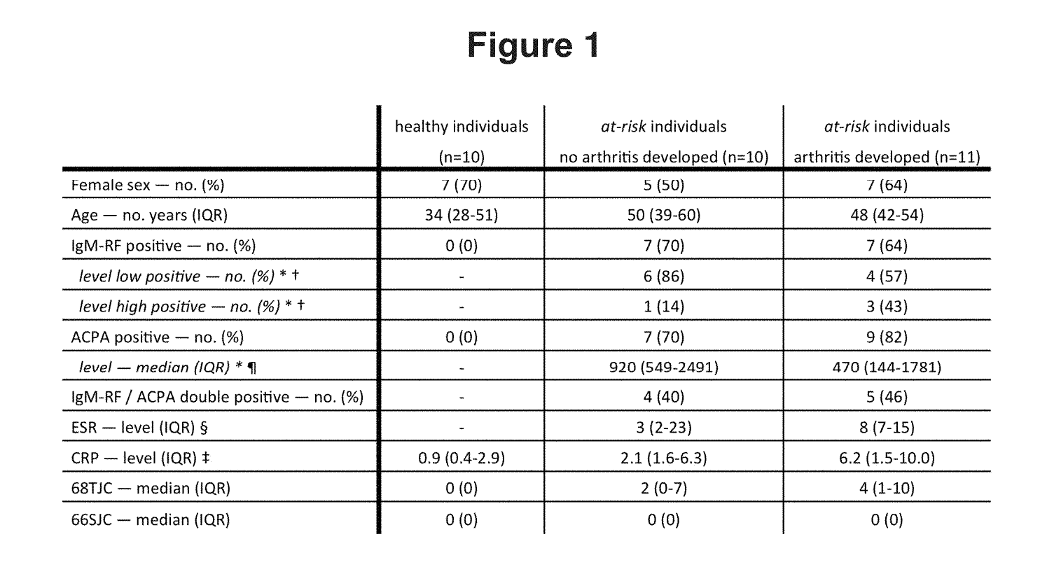

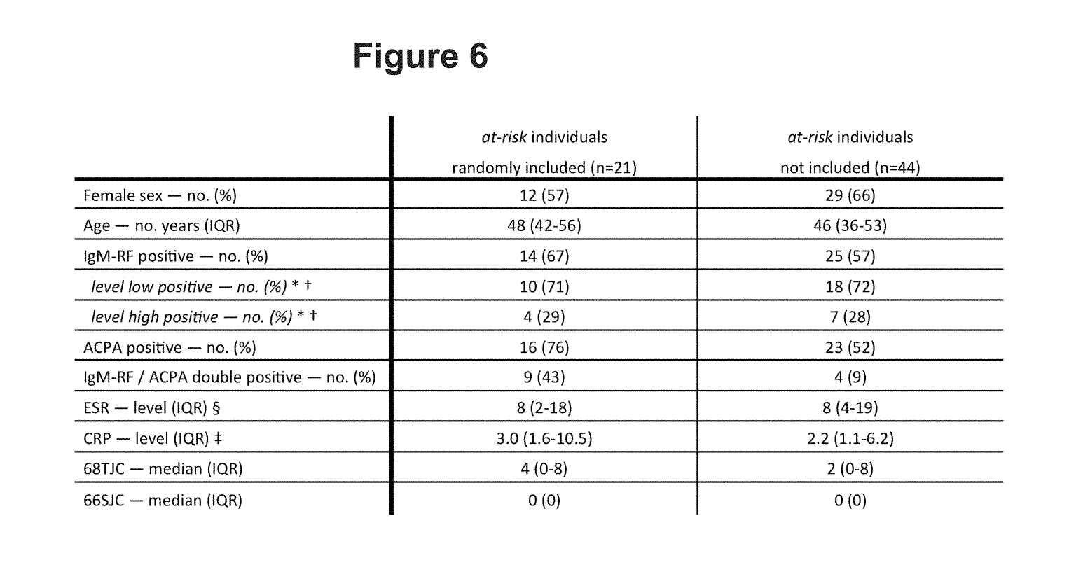

Sixty-five consecutive individuals without arthritis, but at-risk for the development of RA defined by the presence of IgM-RF and/or ACPA were prospectively followed (further denoted as ‘at-risk individuals’) (2, 21). From the 65 included individuals, we randomly selected 10 autoantibody positive at-risk individuals who did not develop arthritis (median follow-up 69 (range 42-78) months), and 11 individuals who did develop arthritis (median follow-up 15 (range 0-65) months) as test cohort. Nine individuals of the latter group fulfilled the 2010 ACR/EULAR criteria for RA at onset of arthritis (22, 23), while two had unclassified arthritis at the moment of development of arthritis but subsequently did fulfill RA criteria over time. In addition, 10 autoantibody negative healthy individuals without any joint complaints were included as controls (clinical characteristics of all groups described in FIG. 1 and FIG. 6). In total 21 at-risk individuals and 10 healthy controls were included for the current study.

A validation cohort was used consisting of 50 consecutively included individuals with elevated ACPA and/or IgM-RF without any signs of arthritis and at least 36 months follow-up (further details are described in (24)). During sequencing and bioinformatic analysis for dominant clones laboratory personnel was blinded for clinical data and outcome.

Peripheral Blood and Synovial Tissue Sampling and Processing

In the 21 at-risk individuals of the test cohort, mini-arthroscopic synovial biopsy sampling was performed upon inclusion in a (non-arthritic) knee joint as previously described (25). Peripheral blood samples were drawn and stored in PAXGene Blood RNA tubes according to the manufacturer's instructions (catalog #762165, PreAnalytiX, Breda, the Netherlands). Storage of synovial biopsies, isolation and quantification of RNA, and cDNA synthesis were performed as described previously (26). The same knee joint biopsied at baseline was selected for mini-arthroscopy in at-risk individuals who subsequently developed arthritis. This was performed just after patient fulfilled the 2010 ACR/EULAR criteria for RA, but before initiation of treatment; during this second arthroscopy one of the biopsied knee joints was clinically inflamed.

Linear Amplification and Next-Generation Sequencing

The linear amplification protocol has been extensively described earlier (26). Details are provided in the supplementary methods. Samples were prepared for sequencing according to the manual for Amplicon Sequencing, and sequenced on a Roche Genome Sequencer FLX (Titanium platform). For each peripheral blood sample 10,000 BCRheavy sequences were analyzed, for each synovial tissue sample 7,500 sequences. Next-generation sequencing (NGS) will visualize expanded B- or plasma cells as a deviation in the repertoire, because they carry the same BCR-sequence. Sequences originating from plasmacytoid cells might be relatively overrepresented in the repertoire, since these cells produce increased amounts of BCR mRNA, contributing to a comparable deviation in the repertoire. We use the term dominant BCR clone to denote clones whose unique BCR signals represent ≥0.5% of the repertoire, as described earlier (20).

Bioinformatics Pipeline and Data Analysis

The bioinformatics pipeline used to obtain the BCR sequences was described previously in detail (27) and contains 4 modules: multiplex identifier (MID)-sorting, identification of V and J gene segments, CDR3 detection and removal of artifacts Immunoglobulin isotype homology was determined using the National Center for Biotechnology Information's open-access web tool Megablast and reference sequences for the human Immunoglobulin heavy-chain constant regions, allowing a sequence homology >97% (28). Values are expressed as mean and standard deviation or median and (interquartile) range, according to criteria for (non-)parametric analysis. Differences between groups were analyzed using Student's t-test, Mann-Whitney U Test, one-way analysis of variance or chi-square test where appropriate. Receiver Operating Characteristic (ROC) curves were used to determine cut-off values for the prediction of arthritis development in the test cohort. Logistic regression analysis was used to predict the added value of high-throughput fingerprinting and quantitation of BCR clones compared to an existing prediction model for the development of RA (24). GraphPad Prism software version 6 and PASW Statistics version 22 were used to perform the analyses. P-values <0.05 were considered statistically significant.

### RESULTS

Identification of Dominant Peripheral Blood BCR Clones Before Onset of Arthritis

Based on earlier observations that dominant BCR clones are present in the synovial tissue during clinically manifest RA, we hypothesized that these clones might be detectable in the peripheral blood before development of arthritis. Indeed, Multiple dominant BCR clones were detected in peripheral blood of all 11 prospectively followed at-risk individuals who developed arthritis from the initial cohort, as long as 66 months before the clinical onset of arthritis. In contrast, dominant BCR clones were nearly absent both in the 10 at-risk individuals who did not develop arthritis and in the 10 healthy individuals (FIG. 2A). We observed that the number of dominant BCR clones, the impact of all dominant BCR clones combined (size of all dominant clones combined as percentage of the total repertoire), and the impact of the most dominant BCR clone were increased in at-risk individuals who developed arthritis, compared to at-risk individuals who did not develop arthritis and healthy individuals (number of dominant clones mean 9.7±8.0 vs. 0.8±0.8 vs. 0.7±0.7 respectively, p=0.001 (FIG. 2B), impact of the dominant clones median 16.4% of the total repertoire, IQR 3.7-33.7% vs. 0.7% IQR 0-1.7% vs. 0.5% IQR 0-1.1% respectively, p<0.0001 (FIG. 2C) and impact of the single most dominant clone mean 5.5%±4.6% vs. 0.7%±0.7% vs. 0.6%±0.4% respectively, p<0.0003 (FIG. 2D)). Subsequently, we analyzed synovial tissue biopsies in at-risk individuals obtained during the pre-clinical phase, but these samples contained BCR mRNA levels that were too low to allow NGS. The low BCR mRNA levels in the synovium during the preclinical stage of the disease are explained by the absence of B cell infiltration using immunohistochemistry (3).

Collectively, these observations demonstrate that expanded BCR clones are readily detectable in peripheral blood during the pre-clinical phase in all at-risk individuals who will develop RA after follow up, but not in those who did not.

The Presence of Dominant BCR Clones Predicts Future Arthritis Development

Having shown that the presence of BCR clones can be detected in peripheral blood in all at-risk individuals who will subsequently develop RA, sometimes after several years, we next aimed to develop a biomarker that can be used to identify individuals who have a high risk of developing arthritis in the short term. Such patients might be treated in the at-risk phase to prevent onset of arthritis (29). A clinically relevant follow-up period of thirty-six months was chosen to evaluate arthritis development. This time period may carry a risk high enough to justify preventive pharmacological intervention, while being short enough to infer urgency for treatment.

We designed three tests based on the number of dominant BCR clones present, the impact of all dominant clones combined on the BCR repertoire, and the impact of the single most dominant BCR clone; ROC curves are depicted in FIG. 4A-C. Based on these ROC curves, optimal cut-offs were determined at dominant BCR clones in peripheral blood, a combined impact 3.7%, and an impact of the most dominant clone 2.5% respectively. We decided to use the presence of dominant BCR clones as comprehensible and intuitive marker for further studies. This is further denoted as ‘BCR-clone positive’, and <5 dominant BCR clones as ‘BCR-clone negative’, and collectively as the BCR-clone model.

The cut-off of ≥5 dominant clones in peripheral blood resulted in two clearly distinguishable groups, and corresponding sensitivity of 78%, specificity of 92%, positive predictive value (PPV) of 72% and negative predictive value (NPV) of 94% (FIG. 4F, and FIG. 4D for the Kaplan-Meier curve, log rank test p<0.001). We had access to a second, independent cohort of 50 subjects to validate our findings using this same cut-off. Fifteen at-risk individuals developed arthritis within 36 months; the characteristics are described in FIG. 3. Analysis in this validation cohort showed that BCR-clone positive at-risk individuals had a 83% risk of developing RA within 36 months (PPV), while this risk was 13% in at-risk BCR-clone negative individuals (1-NPV), resulting in a relative risk of 6.3 (95%-CI 2.7-15, p<0.0001, Kaplan-Meier curve, log rank test p<0.001, FIG. 4E). Post-hoc analysis on both cohorts revealed that within 60 months, all BCR-clone positive individuals developed arthritis (after 47, 48 and 60 months respectively, FIG. 7).

The 50 at-risk individuals in the validation cohort were previously used to develop a prediction model for the development of RA (24), the risk rule model. This describes a composite score of multiple clinical parameters categorizing at-risk individuals into low, intermediate and high-risk individuals (respectively 17, 20 and 13 individuals). Using logistic regression analysis to calculate the added value of the BCR-clone model to the existing risk rule, an overall relative risk of 5.0 (95% CI 1.2-20, p=0.024) was found. In the low, intermediate and high-risk groups the relative risk contributed by BCR-clone positivity was estimated at 18 (95% CI 0.6-520), 6.1 (95% CI 1.9-20) and 1.2 (95% CI 0.6-2.7) respectively.

In conclusion, we show that at-risk individuals with 5 or more dominant BCR clones in peripheral blood have a 83% risk of developing arthritis within 36 months, compared to a risk of 13% in individuals with 4 or less dominant BCR clones. Moreover, analysis after 5 years revealed that all individuals who initially tested positive developed arthritis.

Dominant BCR Clones Present In Peripheral Blood During The Preclinical Phase Have Migrated To Synovial Tissue In Clinically Manifest RA

We hypothesized that if the observed dominant BCR clones are involved in synovial inflammation, that these clones might also be detectable in synovial tissue after onset of RA. To this end we analyzed peripheral blood samples obtained during the preclinical stage, and paired blood and synovial tissue after onset of arthritis in 8 individuals who developed arthritis.

On average, 29% of all pre-clinically detectable dominant peripheral blood BCR clones were detectable in synovial tissue after development of arthritis. All ranked within the top-25 most dominant clones in synovial tissue (FIG. 5A/C). Most strikingly, none of the pre-clinically detectable dominant peripheral blood BCR clones could be recovered from peripheral blood after arthritis developed (FIG. 5B/C). Additional analyses of the dominant clones found in both peripheral blood and synovial tissue showed that these clones are class-switched to the IgG1 isotype and enriched for IGHV4-34, while CDR3 lengths are comparable between the 3 groups, features that are all associated with autoreactivity (described in supplementary results and FIG. 8) (30-32).

Together, these analyses show that dominant BCR peripheral blood clones present during the preclinical phase of arthritis are in part retrievable as dominant clones in synovial tissue once arthritis becomes apparent. At this time point the clones are not found in peripheral blood anymore.

These migratory clones have features that have been associated with autoreactivity.

### DISCUSSION

The results presented here show that the presence of dominant BCR clones in peripheral blood predicts with high accuracy the onset of arthritis in patients who are at-risk of developing RA. Moreover, we found support that these dominant clones may migrate to synovial tissue once arthritis becomes apparent. These findings are consistent with the notion that B cell abnormalities occur up to several years before the onset of synovial inflammation, and that the development of RA is a multistep process. Conceivably, a ‘second hit’, for instance a trauma or viral infection, may lead to synovial inflammation, subsequent migration of autoreactive B-cell clones towards the synovium, and impaired resolution of inflammation in patients with pre-existing systemic autoimmunity (33,34). This work provides the rationale for future studies on B-cell abnormalities during the preclinical stage in other immune-mediated inflammatory diseases like systemic lupus erythematosus, multiple sclerosis and type 1 diabetes, and opens up the perspective of preventive intervention.

Since not all individuals with RA-specific antibodies progress to clinically manifest RA, better biomarkers are needed to predict which at-risk individuals will develop RA. Autoantibody and cytokine profiles, specific gene expression patterns, body mass index, current smoking and autonomic nervous system dysfunction all contribute to the risk of developing RA (35-39). Our data provide a novel biomarker that has superior predictive power compared to other available biomarkers evaluated so far. It increases the accuracy of the previously reported prediction rule for the development of arthritis in autoantibody positive subjects (24).

Identification of at-risk individuals who will develop RA in the short term enables development of early preventive strategies (40,41). Our findings support the rationale for B cells, or the interaction between B cells and T cells as targets for preventive therapy. The cut-off used here (5 or more expanded clones) was chosen to be able to identify subjects with a high risk of developing RA with an acceptable negative predictive value to avoid unnecessary treatment. Whether a preventive pharmacological intervention will be considered acceptable is of course dependent on the benefit: risk profile and the cost-effectiveness of the specific treatment.

As discussed above, there is strong evidence for a role of B cells and plasma cells in the pathogenesis of RA. The development of RA is associated with defects in central and peripheral tolerance leading to autoreactive B-cells (42) and circulating autoantibodies can be detected years before the onset of the disease. It is tempting to speculate that the clones identified in the present study are pathogenic B-cells as 1) they are not detected in healthy controls nor in subjects at risk who do not develop RA after follow up, 2) their dominance suggests activity, 3) the clones have seem to migrate to the inflamed synovial tissue after arthritis development, and 4) these clones show additional features associated with autoreactivity.

All patients had clinically manifest arthritis at the time of the second synovial biopsy but the joint that was biopsied was not clinically inflamed except for one patient. Still, these biopsies showed a diverse repertoire resembling the repertoires of clinically inflamed joints, containing the dominant predictive BCR clones identified in peripheral blood in the pre-clinical phase. This can be explained by the fact that clinically uninvolved joints of established RA patients exhibit histologic signs of synovial inflammation, as we described before (43).

In conclusion, we show the presence of increased BCR clonal signatures in peripheral blood obtained during the preclinical stage of RA, predictive of onset of clinically manifest arthritis after follow up. This may serve as a biomarker that could help guide decisions about pharmacological treatment to prevent the onset of clinically manifest disease. Future work should explore whether B-cell clones might predict onset of disease in other B cell-dependent immune-mediated inflammatory disorders. We also found that these BCR clones disappear from the blood and appear in the target tissue during clinically manifest disease where they may drive autonomous disease progression.

### REFERENCES

1. Nielen M M, van Schaardenburg D, Reesink H W, et al. Specific autoantibodies precede the symptoms of rheumatoid arthritis: a study of serial measurements in blood donors. Arthritis and rheumatism. 2004 Feb; 50(2):380-6.

2. van de Sande M G, de Hair M J, van der Leij C, et al. Different stages of rheumatoid arthritis: features of the synovium in the preclinical phase. Annals of the rheumatic diseases. 2011; 70(5):772-7.

3. de Hair M J, van de Sande M G, Ramwadhdoebe T H, et al. Features of the synovium of individuals at risk of developing rheumatoid arthritis: implications for understanding preclinical rheumatoid arthritis. Arthritis & rheumatology. 2014 Mar; 66(3):513-22.

4. Berger T, Rubner P, Schautzer F, et al. Antimyelin antibodies as a predictor of clinically definite multiple sclerosis after a first demyelinating event. The New England journal of medicine. 2003 Jul 10; 349(2):139-45.

5. Wasserfall C H, Atkinson M A. Autoantibody markers for the diagnosis and prediction of type 1 diabetes. Autoimmunity reviews. 2006 Jul; 5(6):424-8.

6. Eriksson C, Kokkonen H, Johansson M, Hallmans G, Wadell G, Rantapaa-Dahlqvist S. Autoantibodies predate the onset of systemic lupus erythematosus in northern Sweden. Arthritis research & therapy. 2011; 13(1):R30.

7. van Schaik F D, Oldenburg B, Hart A R, et al. Serological markers predict inflammatory bowel disease years before the diagnosis. Gut. 2013 May; 62(5):683-8.

8. Aho K, Heliovaara M, Maatela J, Tuomi T, Palosuo T. Rheumatoid factors antedating clinical rheumatoid arthritis. J Rheumatol. 1991; 18(9):1282-4.

9. Rantapaa-Dahlqvist S, de Jong B A, Berglin E, et al. Antibodies against cyclic citrullinated peptide and IgA rheumatoid factor predict the development of rheumatoid arthritis. Arthritis and rheumatism. 2003; 48(10):2741-9.

10. van der Woude D, Rantapaa-Dahlqvist S, Ioan-Facsinay A, et al. Epitope spreading of the anti-citrullinated protein antibody response occurs before disease onset and is associated with the disease course of early arthritis. Annals of the rheumatic diseases. 2010 Aug; 69(8):1554-61.

11. van de Stadt L A, van der Horst A R, de Koning M H, et al. The extent of the anti-citrullinated protein antibody repertoire is associated with arthritis development in patients with seropositive arthralgia. Annals of the rheumatic diseases. 2011; 70(1):128-33.

12. Deane K D, O'Donnell C I, Hueber W, et al. The number of elevated cytokines and chemokines in preclinical seropositive rheumatoid arthritis predicts time to diagnosis in an age-dependent manner. Arthritis and rheumatism. 2010; 62(11):3161-72.

13. Kokkonen H, Soderstrom I, Rocklov J, Hallmans G, Lejon K, Rantapaa Dahlqvist S. Up-regulation of cytokines and chemokines predates the onset of rheumatoid arthritis. Arthritis and rheumatism. 2010; 62(2):383-91.

14. Schellekens G A, Visser H, de Jong B A, et al. The diagnostic properties of rheumatoid arthritis antibodies recognizing a cyclic citrullinated peptide. Arthritis and rheumatism. 2000; 43(1):155-63.

15. Bos W H, Wolbink G J, Boers M, et al. Arthritis development in patients with arthralgia is strongly associated with anti-citrullinated protein antibody status: a prospective cohort study. Annals of the rheumatic diseases. 2010; 69(3):490-4.

16. Schellekens G A, de Jong B A, van den Hoogen F H, van de Putte L B, van Venrooij W J. Citrulline is an essential constituent of antigenic determinants recognized by rheumatoid arthritis-specific autoantibodies. The Journal of clinical investigation. 1998 Jan 1; 101(1):273-81.

17. Tak P P, Smeets T J, Daha M R, et al. Analysis of the synovial cell infiltrate in early rheumatoid synovial tissue in relation to local disease activity. Arthritis and rheumatism. 1997 Feb; 40(2):217-25.

18. Vossenaar E R, Smeets T J, Kraan M C, Raats J M, van Venrooij W J, Tak P P. The presence of citrullinated proteins is not specific for rheumatoid synovial tissue. Arthritis and rheumatism. 2004 Nov; 50(11):3485-94.

19. Edwards J C, Szczepanski L, Szechinski J, et al. Efficacy of B-cell-targeted therapy with rituximab in patients with rheumatoid arthritis. The New England journal of medicine. 2004 Jun 17; 350(25):2572-81.

20. Doorenspleet M E, Klarenbeek P L, de Hair M J, et al. Rheumatoid arthritis synovial tissue harbours dominant B-cell and plasma-cell clones associated with autoreactivity. Annals of the rheumatic diseases. 2014 Apr; 73(4):756-62.

21. Gerlag D M, Raza K, van Baarsen L G, et al. EULAR recommendations for terminology and research in individuals at risk of rheumatoid arthritis: report from the Study Group for Risk Factors for Rheumatoid Arthritis. Annals of the rheumatic diseases. 2012; 71(5):638-41.

22. Aletaha D, Neogi T, Silman A J, et al. 2010 heumatoid arthritis classification criteria: an American College of Rheumatology/European League Against Rheumatism collaborative initiative. Arthritis and rheumatism. 2010; 62(9):2569-81.

23. Aletaha D, Neogi T, Silman A J, et al. 2010 rheumatoid arthritis classification criteria: an American College of Rheumatology/European League Against Rheumatism collaborative initiative. Annals of the rheumatic diseases. 2010; 69(9):1580-8.

24. van de Stadt L A, Witte B I, Bos W H, van Schaardenburg D. A prediction rule for the development of arthritis in seropositive arthralgia patients. Annals of the rheumatic diseases. 2013; 72(12):1920-6.

25. van de Sande M G, Gerlag D M, Lodde B M, et al. Evaluating antirheumatic treatments using synovial biopsy: a recommendation for standardisation to be used in clinical trials. Annals of the rheumatic diseases. 2011; 70(3):423-7.

26. Klarenbeek P L, de Hair M J, Doorenspleet M E, et al. Inflamed target tissue provides a specific niche for highly expanded T-cell clones in early human autoimmune disease. Annals of the rheumatic diseases. 2012; 71(6):1088-93.

27. Klarenbeek P L, Tak P P, van Schaik B D, et al. Human T-cell memory consists mainly of unexpanded clones. Immunol Lett. 2010; 133(1):42-8.

28. Giudicelli V, Chaume D, Lefranc MP. IMGT/GENE-DB: a comprehensive database for human and mouse immunoglobulin and T cell receptor genes. Nucleic Acids Res. 2005; 33(Database issue):D256-61.

29. Gerlag D M, Norris J M, Tak P P. Towards prevention of autoantibody-positive rheumatoid arthritis: from lifestyle modification to preventive treatment. Rheumatology. 2016; 55(4):607-14.

30. Schroder A E, Greiner A, Seyfert C, et al. Differentiation of B cells in the nonlymphoid tissue of the synovial membrane of patients with rheumatoid arthritis. Proc Natl Acad Sci USA. 1996; 93(1):221-5.

31. Pugh-Bernard A E, Silverman G J, Cappione A J, et al. Regulation of inherently autoreactive VH4-34 B cells in the maintenance of human B cell tolerance. J Clin Invest. 2001; 108(7):1061-70.

32.Wardernann H. Yurasov S, Schaefer A, et al. Predominant autoantibody production by early human B cell precursors. Science 2003; 301(5638):1374-7.

33. Van de Sande M G, de Hair M J, van der Leij C, et al. Different stages of rheumatoid arthritis: features of the synovium in the preclinical phase. Ann Rheum Dis. 2011; 70(5):772-7.

34. Buckley C D, Gilroy D W, Serhan C N, et al. The resolution of inflammation. Nat Rev Immunol. 2013; 13(1):59-66.

35. van Baarsen L G, Bos W H, Rustenburg F, et al. Gene expression profiling in autoantibody-positive patients with arthralgia predicts development of arthritis. Arthritis and rheumatism. 2010; 62(3):694-704.

36. Sokolove J, Bromberg R, Deane K D, et al. Autoantibody epitope spreading in the pre-clinical phase predicts progression to rheumatoid arthritis. PLoS One. 2012; 7(5):e35296.

37. Lubbers J, Vosslamber S, van de Stadt L A, et al. B cell signature contributes to the prediction of RA development in patients with arthralgia. Ann Rheum Dis 2015; 0:1-2. doi:10.1136/annrheumdis-2015-207324.

38. de Hair M J, Landewé R B, van de Sande M G, et al. Smoking and overweight determine the likelihood of developing rheumatoid arthritis. Ann Rheum Dis. 2013; 72(10):1654-8.

39. Koopman F A, Tang M W, Vermeij J, et al. Autonomic dysfunction precedes development of rheumatoid arthritis: a prospective cohort study. EBioMedicine. 2016; 6:231-7.

40. Tak P P. Are we ready to change the pace of arthritis treatment? Treating pre-arthritis and very early arthritis. Acta Reumatol Port. 2011; 36(1):8-9.

41. Gerlag D M, Norris J M, Tak P P. Towards prevention of autoantibody-positive rheumatoid arthritis: from lifestyle modification to preventive treatment. Rheumatology. 2016; 55(4):607-14.

42. Samuels J, Ng Y S, Coupillaud C, Paget D, Meffre E. Impaired early B cell tolerance in patients with rheumatoid arthritis. The Journal of experimental medicine. 2005; 201(10):1659-67.

43. Kraan M C, Versendaal H, Jonker M, et al. Asymptomatic synovitis precedes clinically manifest arthritis. Arthritis and rheumatism. 1998; 41(8):1481-8.

### SUPPLEMENTARY METHODS

Synovial biopsies were snap-frozen in liquid nitrogen and cryopreserved until use. RNA was isolated using a polytron tissue homogenizer (Kinematica AG, Littau-Lucerne, Switzerland) in the presence of STAT60 RNA reagent (Tel-test Inc, Friendswood, Tex.) according to the manufacturer's protocol. After isolation RNA was purified using the RNeasy Mini System (Clean-up-protocol (#74106, Qiagen).

RNA quality was checked using the Bioanalyzer 2100 system (Agilent) and quantified using the Qubit 1.0-platform (#Q32857, Invitrogen Life Technologies, Breda, the Netherlands). cDNA was synthesized using Superscript RT-III and oligo-dT primers according to the manufacturer's protocol (#18080-051, Invitrogen). Linear amplification (LA) of the complete B-cell receptor (BCR) repertoire was performed as described earlier (1). In the first step of LA, cDNA was amplified using a custom primer set. All Vheavy-primers contain the primerB sequence needed for sequencing according to the 454-titanium protocol for amplicon sequencing (version 2010) (Roche Diagnostics, Mannheim, Germany). In the first step of LA the cDNA was amplified in the presence of 5 pmol of each of the V-primers, lx buffer B (Solis BioDyne, Tartu, Estonia), 1 mM MgC12, 0.1 mM dNTPs and 3U of Hotfire (Solis BioDyne) in a volume of 20 uL using a T-Professional thermocycler (Biometra, Goettingen, Germany) (96° C. (900s), 40 cycles (96° C. (30s), 60° C. (60s), 72° C. (60s)), 72° C. (600s)). Amplified products were purified using AMPure XP SPRI-beads (#A63881, Agencourt-Bioscience, Beverly, Mass.) in a template:bead ratio of 0.9. After the first amplification step, a generic PCR was performed to prepare the samples for sequencing. In this second reaction primerB was used as generic forward primer and a generic primer specific for the BCR heavy-joint gene segment or the constant region was used as reverse primer. The reverse primer contains a multiplex identifier and primerA as described in the amplicon-sequencing manual (Roche). The PCR was performed with 50% of the purified LA product in the presence of 10 pmol of each of the primers, lx buffer B, 1 mM MgC12, 0.1 mM dNTPs and 3U of Hotfire in a volume of 40 uL using a T-Professional thermocycler (96° C. (900s), 35 cycles (96° C. (30s), 60° C. (60s), 72° C. (60s)), 72° C. (600s)). After amplification, samples were again purified using the AMPure beads and quantified using fluorospectometry (Quant-iT dsDNA HS Assay Kit (#Q32851, Invitrogen). Samples were prepared for sequencing according to the manufacturer's protocol for Amplicon Sequencing. NGS was performer on a Roche Sequencer FLX using the Titanium platform. For each sample >60,000 (bead-bound) BCR heavy sequences were analyzed.

### SUPPLEMENTARY RESULTS

### Dominant Peripheral Blood BCR Clones Are Predominantly IgG1+ And Have Autoimmune Features

In an earlier study in a different cohort of rheumatoid arthritis (RA) patients the inventors described features of BCR clones dominant in synovial tissue (1). To analyze whether the dominant peripheral blood clones in at-risk individuals who develop arthritis share these same features the inventors compared them to the top 25 peripheral blood clones in both control groups (at-risk individuals who that did not develop arthritis, and autoantibody-negative healthy individuals).

First, the inventors analyzed the isotypes of the BCR clones and found that the dominant clones in at-risk individuals who developed arthritis were significantly more switched to the IgG isotype compared to at-risk individuals who that did not develop arthritis, and autoantibody-negative healthy individuals (both p<0.0001) (FIG. 8A). Accordingly, the number of IgM+ clones was significantly decreased compared to the control groups (at-risk individuals who that did not develop arthritis, and autoantibody-negative healthy individuals, both p<0.0001). The number of IgA+ and IgD+ clones was comparable between the 3 groups. More detailed analysis of the IgG isobtype showed that all subtypes contributed to the IgG predominance, but this was only statistically significant for the IgG1 subtype (p<0.0001) (FIG. 8B).

The inventors reported before that dominant synovial tissue clones are enriched for IGHV4-34 (1), a V_{heavy} gene previously associated with autoreactivity (2). In this study the inventors found that the number of IGHV4-34+ clones was significantly overrepresented in dominant clones in at-risk individuals who developed arthritis compared to at-risk individuals who that did not develop arthritis, and healthy individuals (p=0.008), albeit at low numbers (FIG. 8C). The inventors did not observe an increase in the (amino acid) length of the third complementary determining region (CDR3), which also has been associated with autoantibodies (p=0.89; FIG. 8D). Taken together, dominant clones that are present in the pre-symptomatic phase of RA are class-switched to the IgG1 isotype and enriched for IGHV4-34, while CDR3 lengths are comparable between at-risk individuals that did and did not develop RA or healthy individuals.

### REFERENCES

1. Doorenspleet M E, Klarenbeek P L, de Hair M J, van Schaik B D, Esveldt R E, van Kampen A H, et al. Rheumatoid arthritis synovial tissue harbours dominant B-cell and plasma-cell clones associated with autoreactivity. Annals of the rheumatic diseases. 2014 Apr; 73(4):756-62.

2. Pugh-Bernard A E, Silverman G J, Cappione A J, Villano M E, Ryan D H, Insel R A, et al. Regulation of inherently autoreactive VH4-34 B cells in the maintenance of human B cell tolerance. The Journal of clinical investigation. 2001 Oct; 108(7):1061-70.

### EXAMPLE 2

The experiment as described above for the validation cohort of 50 individuals was repeated and included another 129 patients. In this group 49 developed arthritis in three years, 36 of these were BCR+. Among the individuals that did not develop arthritis 8 in 80 were BCR+.

The results may be summarized as follows:

Positive predictive value 73%

Negative predictive value 98%

Sensitivity 94%

Specificity 87%

Combining the two validations:

Positive predictive value 75%

Negative predictive value 94%

Sensitivity 86%

Specificity 89%

Thus the strong predictive value was replicated in the second validation cohort. Of the 56 individuals who were BCR+ 46 developed arthritis during follow-up, of whom 42 within 3 years.

Time To Arthritis

Interestingly the number of dominant BCR clones, and especially the impact of these dominant BCR clones clearly correlated with the time to arthritis. A higher number of BCR clones indicates a shorter time to arthritis development.

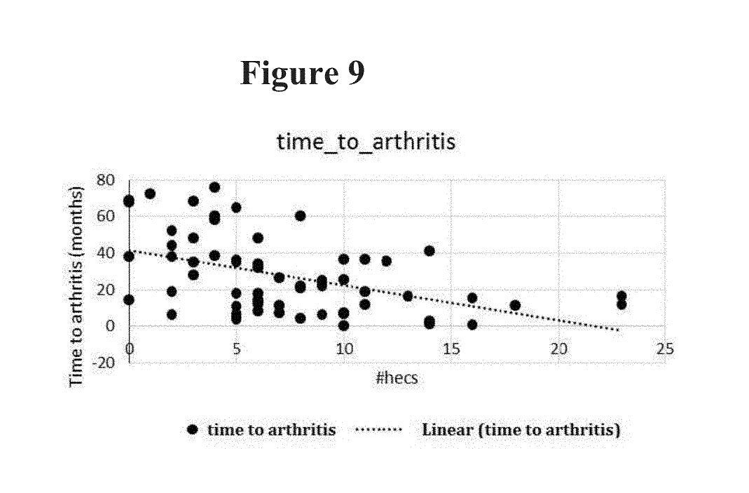

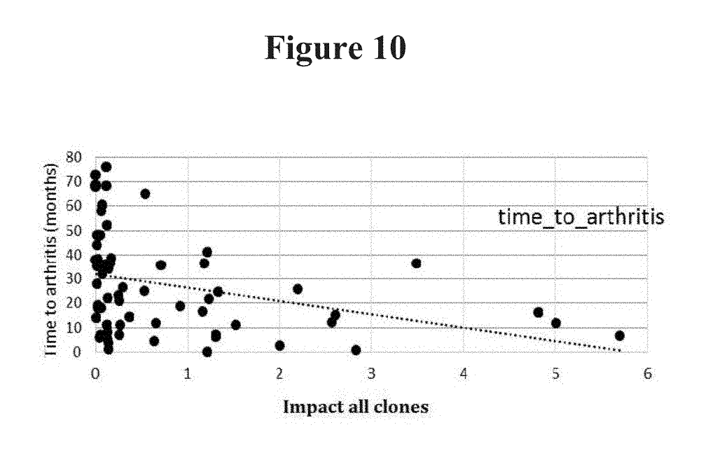

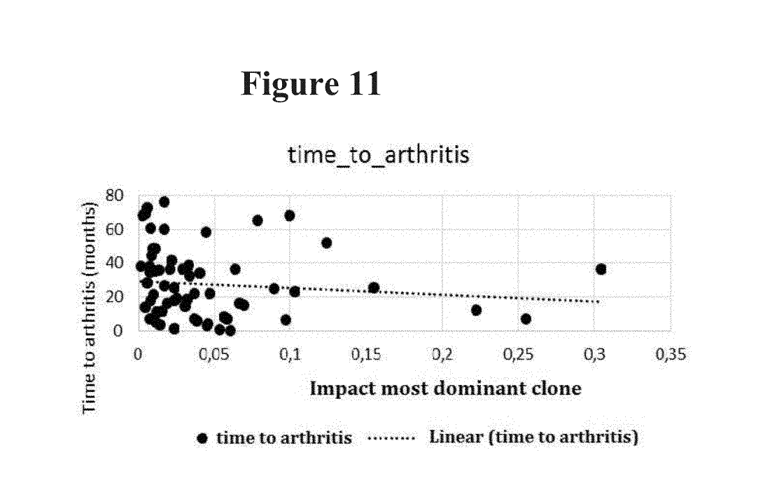

Interestingly, in the individuals that developed arthritis, the time to arthritis clearly correlated with the number of expanded BCR clones (HECs) present (see FIG. 9). This also held for the correlation with total impact of dominant BCR clones (see FIG. 10), and to a lesser extent for the impact of the most dominant BCR clone (see FIG. 11).

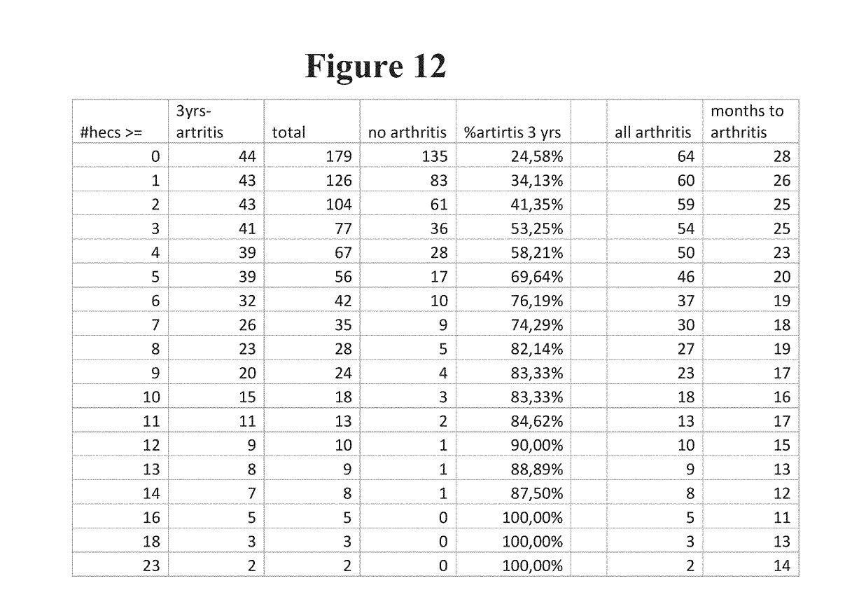

The results are summarized in FIG. 12. In conclusion the risk for arthritis within 3 years is higher if the number of BCR clones is increased. Also arthritis develops earlier. If treatment would be started this would mean that with higher number of BCR clones the benefit/risk ratio increases with increasing number of BCR clones.

Preventive Treatment

A cohort of patients at risk of arthritis was treated with rituximab or placebo. Both groups received corticosteroids during infusion. Overall rituximab led to a delay of 12 months in development of RA.

In this cohort we also performed BCR testing. Rituximab had no effect on incidence of arthritis in the BCR− group (12/31 and 12/33 developed arthritis in the rituximab and placebo-treated group respectively). In the BCR+ group only 1 in 7 (14%) of the rituximab treated individuals developed arthritis, while 3 in 4 (75%) of the placebo treated individuals developed arthritis. Rituximab was clearly effective in the BCR+ at risk patients. For comparison: in the combined replication cohorts 28/56 BCR+ individuals had arthritis within one year of follow-up. Please note that the overall risk of arthritis in the PRAIRI study is high since patients had to be positive for both IgM-RF and ACPA, and had to have a C-reactive protein >=0.6 mg/l.

Changes In B Cell Repertoire After B Cell Depletion In The Earliest Stages Of ACPA+ Arthralgia Patients

Background/Purpose: In example 1 herein we described and validated in an independent cohort that the presence of 5 or more highly expanded clones (i.e. clone >0.5% of the BCR repertoire) in the BCR repertoire (“BCR-positives”) predicts onset in IgM-RF and/or ACPA-positive individuals presenting with arthralgia. Following this lead, here we investigated the following research questions: (1) can arthritis be prevented in BCR-positives by treatment with rituximab?, and (2) how does rituximab affect the BCR repertoire, specifically during arthritis development?

Methods: In 75 at risk individuals from the PRAIRI study blood samples were taken at screening, baseline, after 1 month, 6 months, 1 year and when arthritis occurred. Samples were processed for RNA-based next generation sequencing (NGS). B-cell clones were identified by their unique B-cell receptor heavy-chain sequence, the degree of expansion being expressed as a percentage of the total number of NGS reads.

Results: We identified 5,105,113 clones in 11,637,036 BCR-sequences. Out of 75 at risk individuals 28 developed arthritis during follow-up, 14/41 in the rituximab group, and 14/40 in the control group. In total only 11 individuals were BCR-positive at screening, 7 in the rituximab and 4 in the placebo group. In the rituximab treated group 1 in 7 (14%) developed arthritis, while 3 in 4 (75%) in the placebo group developed arthritis. In the BCR-negative group 12 in 31 individuals treated with rituximab and 12 in 33 individuals treated with placebo developed arthritis. During the baseline visit individuals that would get rituximab treatment on average had 4.1 highly expanded clones (HECs) with an impact on the repertoire of 11.1%. One month after the infusion individuals had 29250 clones and 7 HECs with an impact of 12.2% on the total repertoire. Five months later, six months after the first infusion, we measured 13,816 clones and 15 HECs with an impact of 21.5%. One year after the rituximab infusion the total number of clones remained almost the same (29,080), but the number of HECs decreased to a mean of 2 with an impact on the total repertoire of 9.3%. During development of arthritis individuals treated with rituximab had 6.9 HECs, where individuals in the placebo group only had 1 HEC on average. On the other hand, the impact of the HECs on the total repertoire did hardly differ (25.8% and 21.0% respectively).

Conclusion: Rituximab appears to effectively prevent onset of arthritis in the BCR-positive group. As expected treatment with rituximab influences the BCR repertoire. During arthritis a significantly increased number of HECs is present in at-risk individuals treated with rituximab in comparison to individuals treated with placebo. This suggests that a new BCR repertoire has been formed after the rituximab infusion.

1. Method for determining the risk of developing arthritis or for monitoring a response to a preventive treatment of arthritis in a subject, comprising:
(a) determining in a biological sample from said subject the number of dominant B cell receptor (BCR) clones and/or frequency of at least one dominant BCR clone(s), and
(b) determining said risk of developing arthritis or said response based on said number of dominant BCR clones and/or frequency of said at least one dominant BCR clone(s), wherein a dominant BCR clone is defined as a group of cells expressing the same BCR and wherein
the amount of mRNA encoding the BCR of the cells belonging to said BCR clone constitutes at least 0.1% of the total amount of mRNA encoding a BCR in the biological sample, and
wherein an increase of said number of dominant BCR clones and/or a higher frequency of at least one dominant BCR clone(s) compared to a healthy control indicates an increased risk or a poor response.

2. Method according to claim 1, wherein said subject has presence of IgM- RF and/or ACPA.

3. Method according to claim 1, wherein said subject suffers from arthralgia.

4. Method according to claim 1, wherein at least one dominant BCR clone is of the IgG isotype, optionally of the IgG1 subclass.

5. Method according to claim 1, wherein said biological sample is a peripheral blood sample.

6. Method according to claim 1, wherein an increased risk is indicated when at least 2, 3, 4, 5, 10 or 15 dominant clones are present in said biological sample.

7. Method according to claim 1, wherein said developing arthritis occurs within 60 months, optionally within 48 or 36, 24 or 12 months.

8. Method according to claim 1, wherein said arthritis is rheumatoid arthritis (RA).

9. Method according to claim 3, wherein said rheumatoid arthritis is as defined according to ACR and/or EULAR criteria.

10. Method according to claim 1, comprising:
(a) obtaining a nucleic acid from the biological sample,
(b) performing amplification of representative sequences of the B cell receptor which enable the identification of a B cell receptor,
(c) quantifying a number and/or frequency of B cell receptor(s), and
(d) determining a number and/or frequency of dominant BCR clone(s) based on said number and/or frequency of B cell receptor(s).

11. Method according to claim 10, wherein said representative sequences of the B cell receptor comprise unique VDJ and/or CDR3 sequences of the heavy chain of said B cell receptor, or VJ and/or CDR3 sequences of the light chain of the B cell receptor.

12. Method according to claim 11, wherein the amplification of the nucleic acid is performed using a first primer capable of specifically hybridizing in stringent conditions with the nucleic acids selected from the group consisting of a IGHV, IGKV and IGLV, and a second primer capable of specifically hybridizing in stringent conditions with a nucleic acid selected from IGHC,IGHJ, IGKC, IGKJ, IGLC and IGLJ.

13. Method according to claim 1, wherein said method is used in combination with one or more further biomarker(s) associated with the development of arthritis or rheumatoid arthritis, or with a response to a preventive treatment of arthritis.

14. Compound for use in preventive treatment of a subject at risk of developing arthritis, wherein said risk is determined according to the method according to claim 1.

15. Compound for use according to claim 14, wherein said compound is selected from Rituximab, Etanercept, Adalimumab, Anakinra Infliximab and Abatacept.

16. Compound for use in preventive treatment of a subject at risk of developing arthritis, wherein effectiveness of therapy is predicted according to the method according to claim 1.

17. Compound for use according to claim 16, wherein said compound is selected from Rituximab, Etanercept, Adalimumab, Anakinra Infliximab and Abatacept.

18. Compound for use according to claim 17, wherein said compound is selected a compound targets or depletes B-cells, plasmablasts and/or plasmacells.
